(19) United States
(12) Patent Application Publication
(10) Pub. No.: US 2010/0297265 A1
(43) Pub. Date: Nov. 25, 2010

(54) Optimal compositions and methods for treating oral disease and pain

(76) Inventor: Jeffrey A. McKinney, Lafayette, CA (US)

Correspondence Address: McKinney Law Group APC, 851 Moraga Road, Bungalow B, Lafayette, CA, 94549, US

(21) Appl. No.: 12/800,485

(22) Filed: May 17, 2010

Related U.S. Application Data
(60) Provisional application No. 61/216,664, filed on May 20, 2009.

(51) Int. Cl. A61K 33/10; A61P 43/00

(52) U.S. Cl. 424/717

(57) ABSTRACT

The present invention generally relates to compositions and methods for oral care. In particular it relates to optimal compositions and methods for treating oral disease and pain where the compositions include cesium and/or rubidium. In a composition aspect, the present invention provides a composition for treating oral disease or oral pain. The composition includes cesium bicarbonate, rubidium bicarbonate, or a mixture of cesium bicarbonate and rubidium bicarbonate at a concentration between 40 mM and 110 mM in an aqueous solution. The composition has a pH between 7.0 and 9.0.

This application claims priority from U.S. provisional patent application Ser. No. 61/216,664 filed May 20, 2009, incorporated herein by reference in its entirety.

### FIELD OF THE INVENTION

The present invention generally relates to compositions and methods for oral care. In particular it relates to optimal compositions and methods for treating oral disease and pain where the compositions include cesium and/or rubidium.

### BACKGROUND OF THE INVENTION

Oral disease and oral pain remain important health issues despite over a hundred years of active research by dentists, physicians and scientists. Progress in these areas is oftentimes marked by addressing one or more symptoms rather than eliminating the root cause of the problem.

The treatment of oral disease, e.g., gingivitis, tends to focus on the reduction or elimination of active plaques, i.e., bacterial deposits on the surface of a tooth. The therapies start with patient self-care, which consists of tooth brushing and interstitial hygiene using either dental floss or a toothpick. Professional cleaning is used where significant amounts of plaque have accessed deep periodontal pockets; surgery is usually indicated where professional cleaning does not substantially address the oral disease.

Chemotherapeutic agents are also used to either reduce plaque or prevent it from accumulating. Chlorhexidine, for example, is a cationic agent used for such purposes. These agents may, however, produce unpleasant side effects: Chlorhexidine can cause teeth staining and has an unpleasant taste. Furthermore, chemotherapeutic agents may not adequately address plaque build-up, meaning that they may be of marginal benefit.

There are several over-the-counter medications for the treatment of oral pain. ANBESOL®, for instance, is marketed for the treatment of oral pain resulting from teething, canker sores and denture irritation. PEROXYL® Antiseptic Dental Rinse is marketed to promote oral wound healing and to reduce resulting pain. ORABASE® is used to provide temporary relief of pain created by braces or dentures. BONJELA® Oral Pain-Relieving Gel is applied to relieve teething an mouth ulcer pain, while lignocaine- and xylocaine-based rinses are sold for the treatment of oral mucositis symptoms. As with the chemotherapeutic agents for treating oral disease, however, the over-the-counter oral pain relievers may offer only marginal relief.

There is accordingly a need in the art for optimal compositions and methods for treating oral disease and pain.

### REFERENCES

D. C. Abelson et al., “The effect of saliva on plaque pH in vivo,” J Dent Res, 60(9):1634-8 (September 1981)—Abstract Only.

Akademiia Nauk SSSR, Dobladay, 161(1):244-247 (1965).

E. Bashir et al., Department of Cardiology, School of Dentistry, Karolinska Institutet, Huddinge, Sweden, “Site specificity of citric acid retention after an oral rinse,” Caries Res, 29(6):467-9 (1995)—Abstract Only.

B. G. Bibby et al., “Oral food clearance and the pH of plaque and saliva,” J Am Dent Assoc, 112(3):333-7 (March 1986)—Abstract O D. Birkhed et al., “pH Changes in Human Dental Plaque from Lactose and Milk before and After Adaptation,” Caries Res 27:43-50 (1993)—Abstract Only.

A. H. Brook et al., “A Clinical Study of the Effect of Calcium Glycerophosphate,” Hely. Odont. Acta, 17:55 (October 1973).

C. M. Christensen et al., “Salivary changes in solution pH: a source of individual differences in sour taste perception,” Physiol Behav, 40(2):221-7 (1987)—Abstract Only.

“Changes in Tissue pH and Temperature after Incision Indicate Acidosis May Contribute to Postoperative Pain.” Abstract: Anesthesiology. Volume 101 (2) August 2004 p. 468-475.

M. W. Dodds et al., “Effects of dietary sucrose levels on pH fall and acid-anion profile in human dental plaque after a starch mouth-rinse,” Arch Oral Biol, 31(8):509-12 (1986)—Abstract only.

M. W. Dodds et al., “The relationship between plaque pH, plaque acid anion profiles, and oral carbohydrate retention after ingestion of several ‘reference foods’ by human subjects,” J. Dent Res, 67(5):861-5 (May 1988)—Abstract Only.

F. M. Eggert et al., “The pH of gingival crevices and periodontal pockets in children, teenagers and adults,” Arch Oral Biol, 36(3):233-8 (1991)—Abstract Only.

L. A. Elson et al., “The Sugar Content And The pH Of The Smoke Of Cigarette, Cigar And Pipe Tobaccos In Relation To Lung Cancer,” Int J Cancer, 9(3):666-675 (1972)—Abstract Only.

T. H. Grenby, “Comparison of the Cariostatic Effects of Calcium and Sodium Glycerophosphates in Rats,” Helv. Odont. Acta, 17:54, 55 (October 1973).

G. L. Hays et al., “Salivary pH while dissolving vitamin C-containing tablets,” Am J Dent, 5(5):269-71 (October 1992)—Abstract Only.

S. Kalfas et al, Effect of pH on acid production from sorbitol in washed cell suspensions of oral bacteria, Caries Res, 24(2):107-12 (1990)—Abstract Only.

P. Lingstrom et al., “Effect of frequent consumption of starchy food items on enamel and dentin demineralization and on plaque pH in situ,” J Dent Res, 73(3):652-60 (March 1994)—Abstract Only.

Lyda Associates Inc.® 1990, “Dental erosion by fruit,” Nutrition Research Newsletter, 9(1):6(2) (January 1990)—Abstract Only.

Lyda Associates Inc.® 1990, “Dental properties of soft drinks,” Nutrition Research Newsletter, 9(1:7(1) (January 1990).

L. M. Macpherson et al., “An in vitro stimulation of the effects of chewing sugar-free and sugar-containing chewing gums on pH changes in dental plaque,” J Dent Res, 72(10):1391-7 (October 1993)—Abstract Only.

G. Maglis et al., “Determination of saliva pH in periodontal disease patients and a control group,” Rev Dent Chile, 80(2):70-2 (August 1989)—Abstract Only.

I. D. Mandel, “The role of saliva in maintaining oral homeostasis,” Journal of the American Dental Association, 8(8:298 (7) (1989)—Abstract Only.

A. Millward et al., “Continuous monitoring of salivary flow rate and pH at the surface of the dentition following consumption of acidic beverages,” Caries Res, 31(1):44-9 (1997)—Abstract only.

P. J. Moynihan et al., “A comparison of the relative acidogenic potential of infant milk and soy infant formula: a plaque pH study,” Int J Paediatr Dent, 6(3):177-81 (September 1996)—Abstract Only.

News Bites, Single Sheet, “Sports Drinks: Bad for Teeth?” HealthNews, p. 8 (Apr. 15, 1997).

J. R. Newland, “Oral ulcers: keys to differential and definitive diagnosis,” Consultant, 29(5):157(11) (May 1989).

K. Nilner et al., “Effect of a buffering sugar-free lozenge on intraoral pH and electrochemical action,” Acta Odontol Scand, 49(5):267-72 (October 1991)—Abstract Only.

P. L. Schroeder et al., “Dental erosion and acid reflux disease,” Ann Intern Med, 122(11):809-15 (Jun. 1, 1995).

C. C. Schurer-Maly et al., “Smoking and pH response to H2-receptor antagonists,” Scand J Gastroenterol, 24(10):1172-8 (December 1989)—Abstract Only.

Single page, “Medical Update—Heartburn and Tooth Trouble,” Consumer Reports on Health, p. 106 (September 1995).

Single Sheet, last paragraph, Food Processing, p. 659.

Stehpan, Megan M., “Signals from the Frontlines,” The Scientist, Mar. 28, 2005.

Jamie Talan, Newsday Newspaper Article entitled “Scientists getting nearer to knocking out cavities without using fluoride,” p. D4 (January 1997).

The American Dietetic Association.®, Single Information Sheet, “FIG. 1. Definitions of oral health terms,” (Mar. 19, 1997).

C. J. Thomas et al., “Astringent subqualities in acids,” Chem Senses, 20(6):593-600 (December 1995)—Abstract Only.

P. Vahl et al., “Examination of the pH of saliva in children and juveniles in relation to caries, gingivitis and oral hygiene,” Stomatol DDR, 39(4):253-8 (April 1989).

### SUMMARY OF THE INVENTION

In a composition aspect, the present invention provides a composition for treating oral disease or oral pain. The composition includes cesium bicarbonate, rubidium bicarbonate, or a mixture of cesium bicarbonate and rubidium bicarbonate at a concentration between 40 mM and 110 mM in an aqueous solution. The composition has a pH between 7.0 and 9.0.

In a method aspect, the present invention provides a method of treating oral disease or oral pain. The method includes the steps of contacting a composition with one or more oral tissues of a patient in need of treatment. The composition includes cesium bicarbonate, rubidium bicarbonate, or a mixture of cesium bicarbonate and rubidium bicarbonate at a concentration between 40 mM and 110 mM in an aqueous solution. The composition has a pH between 7.0 and 9.0.

### DETAILED DESCRIPTION OF THE INVENTION

The present invention provides optimal compositions and methods for treating oral disease and/or pain. Such compositions include cesium bicarbonate (i.e., CsHCO₃) and or rubidium bicarbonate (i.e., RbHCO₃).

Compositions may be formulated in any suitable way, including, but not limited to: a dentrifice, oral rinse, mouthwash, toothpaste or cream, tooth powder, dental floss, chewing gum, lozenge, mouth spray, and impregnated toothpicks. The compositions may optionally include humectants, gelling agents, abrasives, fluoride sources, desensitizing agents, flavorings, colorings, sweeteners, preservatives, structuring agents, surfactants, anti-calculus agents and anti-plaque agents.

The compositions of the present invention are typically aqueous solutions or suspensions that include cesium bicarbonate and or rubidium bicarbonate at a total concentration between 10 mM and 200 mM. In other words, the composition would have a cesium bicarbonate concentration between 10 mM and 200 mM, a rubidium bicarbonate concentration between 10 mM and 200 mM, or a combined cesium/rubidium bicarbonate concentration between 10 mM and 200 mM.

In certain cases, the concentration of cesium bicarbonate and/or rubidium carbonate is between 20 mM and 150 mM. In other cases, it is between 25 mM and 125 mM, 30 mM and 120 mM, 35 mM and 115 mM, 40 mM and 110 mM, 45 mM and 105 mM, or 50 mM and 100 mM. In still other cases, the concentration is between 40 mM and 75 mM, 45 mM and 70 mM, or 50 mM and 65 mM. In still other cases, the concentration is about 50 mM.

The pH of aqueous-based compositions of the present invention is typically above 6.5. In certain cases, the pH is above 6.75, 7.0, 7.25, 7.5 or 7.75. In other cases the pH is above 8.0 or 8.25. Oftentimes the pH is below 9.0, 8.75 or 8.5, with some compositions having a pH between 8.25 and 8.5.

Where the cesium bicarbonate and/or rubidium bicarbonate is included in a solid or semi-solid, the weight/weight percentage of cesium/rubidium bicarbonate to total weight of the solid or semi-solid—e.g., gum, lozenge—is between 0.5% and 20%. In certain cases, the weight/weight percentage is between 1% and 15%, 1% and 10%, and 1% and 5%.

The structuring agents are typically used in dentrifices and gums to provide desirable textural properties. A nonlimiting list of structuring agents includes: natural gum binders such as gum tragacanth, xanthan gum, gum karaya and gum arabic, seaweed derivatives, smectite clays such as diatomaceous earths, bentonite or hectorite, calcium apatite, carboxyvinyl polymers and water-soluble cellulose derivatives such as hydroxyethyl cellulose, colloidal magnesium, aluminum silicate and sodium carboxymethyl cellulose. The structuring agent is typically included in an amount of from 0 to 5%, preferably 0 to 3% by weight of the composition.

Nonlimiting examples of fluoride sources are sodium fluoride and stannous fluoride.

Nonlimiting examples of humectants are glycerine, propylene glycol, glyceryl triacetate, sorbitol, xylitol, maltitol, polydextrose, quillaia, lactic acid, and urea.

Nonlimiting examples of abrasives one could have in the composition include: silica abrasives, such as hydrated silicas and silica gels, particularly silica xerogels; alumina; insoluble metaphosphates, such as insoluble sodium metaphosphate; calcium carbonate; dicalcium phosphate (in dihydrate and anhydrous forms); and calcium pyrophosphate (including beta-phase calcium). Calcium carbonate is a preferred abrasive. Abrasives are typically included in an amount of from 0-80%, preferably 0-60%, more preferably 5-25% by weight of the oral hygiene composition.

Examples of flavoring agents that may be contained in the composition, include, without limitation, the following: wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavender oil, mustard oil, pine oil, pine needle oil, rosemary oil, thyme oil, cinnamon leaf oil; D-tryptophan; dextrose; levulose; acesulfam; dihydrochalcones; sodium cyclamate; and mixtures thereof. When included in the composition of the present invention, flavoring or sweetening agents are typically included in the oral hygiene composition in an amount from 0-5% by weight, preferably 0-2% by weight.

Coloring agents such as the following may optionally be included in the composition: dyes such as FD & C blue No. 1, D & C yellow No. 10 and D & C yellow No. 3; and titanium dioxide.

Further optional components for use in the compositions include: antioxidants; vitamins (e.g., vitamin C and E); anti-plaque agents (e.g., stannous salts, copper salts, magnesium salts, chlorhexidine, cetylpyridinium chloride, sodium lauryl sulfate, and Tween (polysorbate 20)); pH adjusting agents (e.g., citric acid); anticaries agents (e.g., urea, calcium glycerophosphate, and sodium trimetaphosphate), plant extracts; desensitizing agents for sensitive teeth (e.g., cesium nitrate, cesium citrate, stannous fluoride and potassium oxalate); whitening agents (e.g., carbamide peroxide) and mixtures thereof.

Where the composition of the present invention is used as a denture cleanser, it may additionally include one or more bleaching agents, effervescence generators, and chelating agents. The bleaching agent is typically an inorganic persalt. Examples bleaching agents include, without limitation, the following: alkali metal and ammonium persulphates, perborates, percarbonates, perphosphates, and the alkali metal ions and alkaline earth metal peroxides—e.g., potassium, ammonium, sodium and cesium persulphates and perborate mono- and tetrahydrates, sodium pyrophosphate peroxyhydrate and magnesium, calcium, and zinc peroxides and mixtures thereof.

Denture cleansing compositions of the present invention may be in the form of pastes, tablets, granules or powders.

Compositions of the present invention may also be used in conjunction with hemostatic dental compositions. The combination of hemostatic dental compositions and compositions of the present invention stop oral bleeding and provide gingival tissue fluid control without opening up the dentinal tubules in dentin. Furthermore, by using the subject compositions during dental restorative and reconstructive procedures, bleeding can be stopped so that an accurate impression for a dental prosthetic can be made. The conformational tolerance of the impression mold is significantly increased.

Nonlimiting examples of compositions of the present invention are compositions comprising:

### EXAMPLE 1

Active Ingredient: CsHCO₃
Concentration of Active Ingredient: between 45 mM and 105 mM
pH of Composition: between 7.0 and 9.0
Other Ingredients: water
Form of composition: oral rinse

### EXAMPLE 2

Active Ingredient: CsHCO₃
Concentration of Active Ingredient: between 40 mM and 75 mM
pH of Composition: between 7.0 and 9.0
Other Ingredients: water
Form of Composition: oral rinse

### EXAMPLE 3

Active Ingredient: CsHCO₃
Concentration of Active Ingredient: between 40 mM and 75 mM
pH of Composition: between 7.5 and 9.0
Other Ingredients: water
Form of Composition: oral rinse

### EXAMPLE 4

Active Ingredient: CsHCO₃
Concentration of Active Ingredient: between 40 mM and 75 mM
pH of Composition: between 7.5 and 9.0
Other Ingredients: water
Form of Composition: oral rinse

### EXAMPLE 5

Active Ingredient: CsHCO₃
Concentration of Active Ingredient: between 40 mM and 75 mM
pH of Composition: between 8.0 and 9.0
Other Ingredients: water
Form of Composition: oral rinse

### EXAMPLE 6

Active Ingredient: CsHCO₃
Concentration of Active Ingredient: between 45 mM and 70 mM
pH of Composition: between 8.25 and 8.5
Other Ingredients: water
Form of Composition: oral rinse

### EXAMPLE 7

Active Ingredient: CsHCO₃
Concentration of Active Ingredient: about 50 mM
pH of Composition: between 8.25 and 8.5
Other Ingredients: water
Form of Composition: oral rinse

### EXAMPLE 8

Active Ingredient: CsHCO₃
Concentration of Active Ingredient: 0.5% to 20%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 9

Active Ingredient: CsHCO₃
Concentration of Active Ingredient: 0.5% to 20%
Other Ingredients: xanthan gum, peppermint, eucalyptol, thymol, wintergreen, xylitol, sodium bicarbonate, and zinc
Form of Composition: lozenge

### EXAMPLE 10

Active Ingredient: RbHCO₃
Concentration of Active Ingredient: between 45 mM and 105 mM
pH of Composition: between 7.0 and 9.0
Other Ingredients: water
Form of composition: oral rinse

### EXAMPLE 11

Active Ingredient: RbHCO₃
Concentration of Active Ingredient: between 40 mM and 75 mM
pH of Composition: between 7.0 and 9.0
Other Ingredients: water
Form of Composition: oral rinse

### EXAMPLE 12

Active Ingredient: RbHCO₃
Concentration of Active Ingredient: between 40 mM and 75 mM
pH of Composition: between 7.5 and 9.0
Other Ingredients: water
Form of Composition: oral rinse

### EXAMPLE 13

Active Ingredient: RbHCO₃
Concentration of Active Ingredient: between 40 mM and 75 mM
pH of Composition: between 7.5 and 9.0
Other Ingredients: water
Form of Composition: oral rinse

### EXAMPLE 14

Active Ingredient: RbHCO₃
Concentration of Active Ingredient: between 40 mM and 75 mM
pH of Composition: between 8.0 and 9.0
Other Ingredients: water
Form of Composition: oral rinse

### EXAMPLE 15

Active Ingredient: RbHCO₃
Concentration of Active Ingredient: between 45 mM and 70 mM
pH of Composition: between 8.25 and 8.5
Other Ingredients: water
Form of Composition: oral rinse

### EXAMPLE 16

Active Ingredient: RbHCO₃
Concentration of Active Ingredient: about 50 mM
pH of Composition: between 8.25 and 8.5
Other Ingredients: water
Form of Composition: oral rinse

### EXAMPLE 17

Active Ingredient: RbHCO₃
Concentration of Active Ingredient: 0.5% to 20%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 18

Active Ingredient: RbHCO₃
Concentration of Active Ingredient: 0.5% to 20%
Other Ingredients: xanthan gum, peppermint, eucalyptol, thymol, wintergreen, xylitol, sodium bicarbonate, and zinc
Form of Composition: gum

### EXAMPLE 19

Active Ingredient: CsHCO₃/RbHCO₃
Concentration of Active Ingredient: between 45 mM and 105 mM
pH of Composition: between 7.0 and 9.0
Other Ingredients: water
Form of composition: oral rinse

### EXAMPLE 20

Active Ingredient: CsHCO₃/RbHCO₃
Concentration of Active Ingredient: between 40 mM and 75 mM
pH of Composition: between 7.0 and 9.0
Other Ingredients: water
Form of Composition: oral rinse

### EXAMPLE 21

Active Ingredient: CsHCO₃/RbHCO₃
Concentration of Active Ingredient: between 40 mM and 75 mM
pH of Composition: between 7.5 and 9.0
Other Ingredients: water
Form of Composition: oral rinse

### EXAMPLE 22

Active Ingredient: CsHCO₃/RbHCO₃
Concentration of Active Ingredient: between 40 mM and 75 mM
pH of Composition: between 7.5 and 9.0
Other Ingredients: water
Form of Composition: oral rinse

### EXAMPLE 23

Active Ingredient: CsHCO₃/RbHCO₃
Concentration of Active Ingredient: between 40 mM and 75 mM
pH of Composition: between 8.0 and 9.0
Other Ingredients: water
Form of Composition: oral rinse

### EXAMPLE 24

Active Ingredient: CsHCO₃/RbHCO₃
Concentration of Active Ingredient: between 45 mM and 70 mM
pH of Composition: between 8.25 and 8.5
Other Ingredients: water
Form of Composition: oral rinse

### EXAMPLE 25

Active Ingredient: CsHCO₃/RbHCO₃
Concentration of Active Ingredient: about 50 mM
pH of Composition: between 8.25 and 8.5
Other Ingredients: water
Form of Composition: oral rinse

### EXAMPLE 26

Active Ingredient: CsHCO₃/RbHCO₃
Concentration of Active Ingredient: 0.5% to 20%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 27

Active Ingredient: CsHCO₃/RbHCO₃
Concentration of Active Ingredient: 0.5% to 20%
Other Ingredients: xanthan gum, peppermint, eucalyptol, thymol, wintergreen, xylitol, sodium bicarbonate, and zinc
Form of Composition: gum

### EXAMPLE 28

Active Ingredient: CsHCO₃
Concentration of Active Ingredient: between 45 mM and 105 mM
pH of Composition: between 7.0 and 9.0
Other Ingredients: water, flavoring
Form of composition: oral rinse

### EXAMPLE 29

Active Ingredient: CsHCO₃
Concentration of Active Ingredient: between 40 mM and 75 mM
pH of Composition: between 7.0 and 9.0
Other Ingredients: water, flavoring
Form of Composition: oral rinse

### EXAMPLE 30

Active Ingredient: CsHCO₃
Concentration of Active Ingredient: between 40 mM and 75 mM
pH of Composition: between 7.5 and 9.0
Other Ingredients: water, flavoring
Form of Composition: oral rinse

### EXAMPLE 31

Active Ingredient: CsHCO₃
Concentration of Active Ingredient: between 40 mM and 75 mM
pH of Composition: between 7.5 and 9.0
Other Ingredients: water, flavoring
Form of Composition: oral rinse

### EXAMPLE 32

Active Ingredient: CsHCO₃
Concentration of Active Ingredient: between 40 mM and 75 mM
pH of Composition: between 8.0 and 9.0
Other Ingredients: water, flavoring
Form of Composition: oral rinse

### EXAMPLE 33

Active Ingredient: CsHCO₃
Concentration of Active Ingredient: between 45 mM and 70 mM
pH of Composition: between 8.25 and 8.5
Other Ingredients: water, flavoring
Form of Composition: oral rinse

### EXAMPLE 34

Active Ingredient: CsHCO₃
Concentration of Active Ingredient: about 50 mM
pH of Composition: between 8.25 and 8.5
Other Ingredients: water, flavoring
Form of Composition: oral rinse

### EXAMPLE 35

Active Ingredient: CsHCO₃
Concentration of Active Ingredient: 0.5% to 20%
Other Ingredients: ethyl cellulose polymer, peppermint oil, menthol, gum arabic, sucralose, xylitol, sodium bicarbonate, eucalyptus oil, thymol, wintergreen, glycerol
Form of Composition: lozenge

### EXAMPLE 36

Active Ingredient: CsHCO₃
Concentration of Active Ingredient: 0.5% to 20%
Other Ingredients: xanthan gum, peppermint, eucalyptol, thymol, wintergreen, xylitol, sodium bicarbonate
Form of Composition: gum

### EXAMPLE 37

Active Ingredient: CsHCO₃
Concentration of Active Ingredient: between 45 mM and 105 mM
pH of Composition: between 7.0 and 9.0
Other Ingredients: water, one or more anti-plaque agents selected from chlorhexidine, cetylpyridinium chloride, sodium lauryl sulfate and polysorbate 20
Form of composition: oral rinse

### EXAMPLE 38

Active Ingredient: CsHCO₃
Concentration of Active Ingredient: between 40 mM and 75 mM
pH of Composition: between 7.0 and 9.0
Other Ingredients: water, one or more anti-plaque agents selected from chlorhexidine, cetylpyridinium chloride, sodium lauryl sulfate and polysorbate 20
Form of Composition: oral rinse

### EXAMPLE 39

Active Ingredient: CsHCO₃
Concentration of Active Ingredient: between 40 mM and 75 mM
pH of Composition: between 7.5 and 9.0
Other Ingredients: water, one or more anti-plaque agents selected from chlorhexidine, cetylpyridinium chloride, sodium lauryl sulfate and polysorbate 20
Form of Composition: oral rinse

### EXAMPLE 40

Active Ingredient: CsHCO₃
Concentration of Active Ingredient: between 40 mM and 75 mM
pH of Composition: between 7.5 and 9.0
Other Ingredients: water, one or more anti-plaque agents selected from chlorhexidine, cetylpyridinium chloride, sodium lauryl sulfate and polysorbate 20
Form of Composition: oral rinse

### EXAMPLE 41

Active Ingredient: CsHCO₃
Concentration of Active Ingredient: between 40 mM and 75 mM
pH of Composition: between 8.0 and 9.0
Other Ingredients: water, one or more anti-plaque agents selected from chlorhexidine, cetylpyridinium chloride, sodium lauryl sulfate and polysorbate 20
Form of Composition: oral rinse

### EXAMPLE 42

Active Ingredient: CsHCO₃
Concentration of Active Ingredient: between 45 mM and 70 mM
pH of Composition: between 8.25 and 8.5
Other Ingredients: water, one or more anti-plaque agents selected from chlorhexidine, cetylpyridinium chloride, sodium lauryl sulfate and polysorbate 20
Form of Composition: oral rinse

### EXAMPLE 43

Active Ingredient: CsHCO₃
Concentration of Active Ingredient: about 50 mM
pH of Composition: between 8.25 and 8.5
Other Ingredients: water, one or more anti-plaque agents selected from chlorhexidine, cetylpyridinium chloride, sodium lauryl sulfate and polysorbate 20
Form of Composition: oral rinse

### EXAMPLE 44

Active Ingredient: CsHCO₃
Concentration of Active Ingredient: between 45 mM and 105 mM
pH of Composition: between 7.0 and 9.0
Other Ingredients: water, flavoring, one or more anti-plaque agents selected from chlorhexidine, cetylpyridinium chloride, sodium lauryl sulfate and polysorbate 20
Form of composition: oral rinse

### EXAMPLE 45

Active Ingredient: CsHCO₃
Concentration of Active Ingredient: between 40 mM and 75 mM
pH of Composition: between 7.0 and 9.0
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 46

Active Ingredient: CsHCO₃
Concentration of Active Ingredient: between 40 mM and 75 mM
pH of Composition: between 7.5 and 9.0
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 47

Active Ingredient: CsHCO₃
Concentration of Active Ingredient: between 40 mM and 75 mM
pH of Composition: between 7.5 and 9.0
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 48

Active Ingredient: CsHCO₃
Concentration of Active Ingredient: between 40 mM and 75 mM
pH of Composition: between 8.0 and 9.0
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 49

Active Ingredient: CsHCO₃
Concentration of Active Ingredient: between 45 mM and 70 mM
pH of Composition: between 8.25 and 8.5
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 50

Active Ingredient: CsHCO₃
Concentration of Active Ingredient: about 50 mM
pH of Composition: between 8.25 and 8.5
Other Ingredients: water, flavoring, one or more anti-plaque agents selected from chlorhexidine, cetylpyridinium chloride, sodium lauryl sulfate and polysorbate 20
Form of Composition: oral rinse

### EXAMPLE 51

Active Ingredient: CsHCO₃
Concentration of Active Ingredient: 0.5% to 20%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate, carbamide peroxide
Form of Composition: lozenge

### EXAMPLE 52

Active Ingredient: CsHCO₃
Concentration of Active Ingredient: 0.5% to 20%
Other Ingredients: xanthan gum, peppermint, eucalyptol, thymol, wintergreen, xylitol, sodium bicarbonate, zinc, carbamide peroxide
Form of Composition: lozenge

### EXAMPLE 53

Active Ingredient: CsHCO₃
Concentration of Active Ingredient: 0.5% to 20%
Other Ingredients: carbamide peroxide, peppermint oil, eucalyptol, thymol, wintergreen, xylitol, sodium bicarbonate, zinc
Form of Composition: lozenge

### EXAMPLE 54

Active Ingredient: CsHCO₃
Concentration of Active Ingredient: 1.0% to 15%
Other Ingredients: carbamide peroxide, peppermint oil, eucalyptol, thymol, wintergreen, xylitol, sodium bicarbonate, zinc
Form of Composition: lozenge

### EXAMPLE 55

Active Ingredient: CsHCO₃
Concentration of Active Ingredient: 1.0% to 10%
Other Ingredients: carbamide peroxide, peppermint oil, eucalyptol, thymol, wintergreen, xylitol, sodium bicarbonate, zinc
Form of Composition: lozenge

### EXAMPLE 54

Active Ingredient: CsHCO₃
Concentration of Active Ingredient: 1.0% to 5%
Other Ingredients: carbamide peroxide, peppermint oil, eucalyptol, thymol, wintergreen, xylitol, sodium bicarbonate, zinc
Form of Composition: lozenge

### EXAMPLE 55

Active Ingredient: CsHCO₃
Concentration of Active Ingredient: 1.0% to 15%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 56

Active Ingredient: CsHCO₃
Concentration of Active Ingredient: 1.0% to 15%
Other Ingredients: xanthan gum, peppermint, eucalyptol, thymol, wintergreen, xylitol, sodium bicarbonate, zinc
Form of Composition: lozenge

### EXAMPLE 57

Active Ingredient: CsHCO₃
Concentration of Active Ingredient: 1.0% to 10%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 58

Active Ingredient: CsHCO₃
Concentration of Active Ingredient: 1.0% to 10%
Other Ingredients: xanthan gum, peppermint, eucalyptol, thymol, wintergreen, xylitol, sodium bicarbonate, zinc
Form of Composition: lozenge

### EXAMPLE 59

Active Ingredient: CsHCO₃
Concentration of Active Ingredient: 1.0% to 5%
Other Ingredients: ethyl cellulose polymer, peppermint oil, menthol, gum arabic, sucralose, xylitol, sodium bicarbonate, eucalyptus oil, thymol, wintergreen, glycerol, zinc gluconate
Form of Composition: lozenge

### EXAMPLE 60

Active Ingredient: CsHCO₃
Concentration of Active Ingredient: 1.0% to 5%
Other Ingredients: xanthan gum, peppermint, eucalyptol, thymol, wintergreen, xylitol, sodium bicarbonate, zinc
Form of Composition: lozenge

### EXAMPLE 61

Active Ingredient: CsHCO₃
Concentration of Active Ingredient: 0.5% to 20%
Other Ingredients: abrasive
Form of Composition: dentrifice

### EXAMPLE 62

Active Ingredient: CsHCO₃
Concentration of Active Ingredient: 0.5% to 20%
Other Ingredients: abrasive selected from hydrated silicas and silica gels, (e.g., silica xerogels), alumina, insoluble metaphosphates (e.g., sodium metaphosphate), calcium carbonate, dicalcium phosphate, and calcium pyrophosphate
Form of Composition: dentrifice

### EXAMPLE 63

Active Ingredient: CsHCO₃
Concentration of Active Ingredient: 1.0% to 15%
Other Ingredients: abrasive selected from hydrated silicas and silica gels, (e.g., silica xerogels), alumina, insoluble metaphosphates (e.g., sodium metaphosphate), calcium carbonate, dicalcium phosphate, and calcium pyrophosphate
Form of Composition: dentrifice

### EXAMPLE 64

Active Ingredient: CsHCO₃
Concentration of Active Ingredient: 1.0% to 10%
Other Ingredients: abrasive selected from hydrated silicas and silica gels, (e.g., silica xerogels), alumina, insoluble metaphosphates (e.g., sodium metaphosphate), calcium carbonate, dicalcium phosphate, and calcium pyrophosphate
Form of Composition: dentrifice

### EXAMPLE 65

Active Ingredient: CsHCO₃
Concentration of Active Ingredient: 1.0% to 5%
Other Ingredients: abrasive selected from hydrated silicas and silica gels, (e.g., silica xerogels), alumina, insoluble metaphosphates (e.g., sodium metaphosphate), calcium carbonate, dicalcium phosphate, and calcium pyrophosphate
Form of Composition: dentrifice

Methods of the present invention involve bringing a composition according to the present invention into contact with the oral tissue of a human or animal patient in need of treatment. As noted above, the composition brought into contact with the patient's oral tissue can take many forms including, but not limited to, the following: a dentrifice, oral rinse, mouthwash, toothpaste or cream, tooth powder, dental floss, chewing gum, lozenge, mouth spray, and impregnated toothpicks.

Experiments Related to the Invention

Materials and Methods

Streptococcus mutans strain ATCC 25175 was obtained from American Type Culture Collection in the freeze-dried state. Cells were rehydrated in a trypticase soy-yeast extract media (30 g trypticase soy broth and 3 g yeast extract per liter mqH₂O; TS media). The bacteria were grown overnight at 37° C., shaking at 60 rpm in 50 mL broth. For routine propagation of the strain, S. mutans was grown on trypticase soy agar plates, TS media with 15 g/L agar. Escherichia coli strain K12 was grown as a representative enteric bacteria in LB broth (10 g tryptone, 5 g yeast extract and 10 g NaCl per liter mqH₂O).

To assess the toxicity of cesium chloride, growth rates were calculated from cultures grown in the absence and presence of cesium chloride with sodium chloride added to controls to maintain consistent ionic strength. The first growth curves conducted used duplicate cultures of 50 mL TS media in 125 mL flasks with foam stoppers. The media was inoculated with 1 mL of the starter culture (2% inoculum) and the optical density was measured at 600 nm. Two conditions were initially tested: 100 mM CsCl and 100 mM NaCl.

An increasing gradient of CsCl concentrations was examined to see how bacterial growth rate varies with salt concentration. Concentrations of 0 mM, 5 mM, 10 mM, 25 mM, 50 mM, 75 mM and 100 mM CsCl were tested; NaCl was used to counter balance control in order to maintain constant ion concentrations. Additional gradient growth curves with 0 mM, 20 mM, 30 mM, 40 mM and 50 mM CsCl were conducted to refine the inhibition profile of S. mutans. For comparison, the growth rate of E. coli K12 was observed in the presence of CsCl at the following concentrations: 0 mM, 5 mM, 10 mM, 25 mM, 75 mM, and 100 mM CsCl in LB broth.

To mimic the culture conditions of oral bacteria in the mouth S. mutans was also grown as a biofilm on glass slides in TS liquid media supplemented with 20 mM glucose, shaking at 25 rpm to allow both adherence of the cells and aeration of the media. Slides were cleaned according to the protocol of Svensäter (2007). They were boiled in a 5:1:1 mixture of H₂O:H₂O₂:NH₃ for 15 min., rinsed in deionized H₂O, boiled in a 5:1:1 mixture of H₂O:H₂O₂:HCl for 15 min, and rinsed once with deionized H₂O followed by twice with 99.9% ethanol.

Once cleaned, the slides were sterilized in 250 mL flasks or 50 mL falcon tubes with foam stoppers. They were placed in grown culture for approximately 40 min. to allow adherence of cells to the slides. The slides were quickly dipped in sterile deionized H2O to remove planktonic cells and placed in 250 mL flasks with 100 mL TS media amended with 20 mM glucose. Biofilm growth was quantified via dry weight measurements by scraping the biofilm with a sterile razor into a pre-weighed 50 mL falcon tube and then freeze-drying. Biofilm experiments were conducted with either 100 mM CsCl or 100 mM NaCl added initially or by spiking cultures one day after inoculation with 100 mM CsCl or 100 mM NaCl.

Additional cesium salts were used to assess alternate inhibitors of S. mutans cultures. Cesium salts of hydroxide, formate, bicarbonate, sulfate, nitrate, fluoride, and iodide were obtained from Fisher Scientific. Growth rates of S. mutans were monitored with 0 mM, 25 mM, 50 mM, and 100 mM cesium salt in TS broth. Control cultures were grown with sodium hydroxide, bicarbonate, nitrate, and sulfate at 0 mM, 25 mM, 50 mM, and 100 mM. Inhibition from cesium bicarbonate and sodium bicarbonate was further refined at concentrations of 0 mM, 15 mM, 30 mM, and 45 mM.

In order to better understand the mechanism of inhibition, the following potassium channel mutants of E. coli from the Keio collection were obtained from the Coli Genetic Stock Center at Yale University: Kch mutant JW 1242-1, TrkG mutant JW 1358-1, TrkH mutant JW 5576-1, and KdpA mutant JW686-5. Mutant E. coli strains were grown in LB broth in the presence or absence of CsCl (either 0 mM, 50 mM, or 100 mM CsCl) to assess the role of potassium transport channels in cesium inhibition.

Since S. mutans biofilms have been shown to create a localized area of decreased pH on the tooth surface, the growth of S. mutans was monitored under differing pH conditions to see how the acidic or basic environment can affect the toxicity of cesium chloride. Five different pH levels of media were tested (pH 4.04, 5,60, 7.00, and 8.33) with varying CsCl concentrations of 0 mM, 25 mM and 100 mM.

To determine how CsCl affects exponentially growing S. mutans cells, cultures were spiked with CsCl. TS media was prepared as in previous experiments and cultures were started with 0.2% inoculum. The cultures were grown for three and a half hours while monitoring the growth with the OD at 600 nm. When the cultures were in early exponential growth phase, they were spiked with 100 mM CsCl, 100 mM NaCl, or Listerine mouthwash in a 1:10 or 1:100 dilution in TS media.

Results

To investigate the inhibition of S. mutans by cesium chloride, growth in the presence and absence of CsCl was compared. Ions were balanced with NaCl in all cultures to a final concentration of 100 mM of supplemental cations added to the media (i.e., for 75 mM CsCl, 25 mM NaCl was added to bring the total concentration of supplemental cation to 100 mM). All data is presented in optical density absorbance measurements at 600 nm, or doubling times (the length of time it takes for a culture to double in time).

In cultures with 100 mM NaCl, S. mutans had favorable growth (with a doubling time of roughly 1 hour, presented as 0 mM CsCl in the following data), whereas in a 100 mM CsCl culture the growth of S. mutans was almost completely inhibited. This culture exhibited flocky aggregates of cells without exhibiting normal signs of growth (increasing in overall turbidity of the culture). Since this did not appear to be normal growth, 50 μL of each culture was spread onto a TS-agar plate to analyze their growth. While the NaCl control grew small opaque white colonies overnight, the CsCl culture produced thin translucent colonies after a week, which indicated a stressful growth environment.

In Cs gradient experiments, S. mutans was grown in the presence of 0 mM, 5 mM, 10 mM, 25 mM, 50 mM, 75 mM and 100 mM CsCl. The 0 mM, 5 mM, 10 mM and 25 mM lots grew with similar growth rates and absorbance yields. The calculated doubling times were 1.06 h, 1.06 h, 0.94 h, and 1.17 h respectively. The four different cultures grew to the same maximum yield of 0.9 OD units, which indicates cesium has little effect on the total growth and yield of cells at those concentrations.

At higher CsCl concentrations (i.e., 50 mM, 75 mM and 100 mM), the doubling times increased to 1.54 h, 2.37 h and 4.17 h, respectively, indicating that higher concentrations inhibit the growth of S. mutans. Further cultures at concentrations of 0 mM, 20 mM, 30 mM, 40 mM and 50 mM were tested to refine the data. A Lineweaver-Burke plot of the data indicates the Minimum Inhibitory Concentration (i.e., MIC) for CsCl is 61.2 mM. Concentrations of cesium below the MIC appear to have little effect on bacterial growth rate, while higher concentrations have varying effects on the rate of growth and final yields.

To determine whether CsCl would have an adverse effect on enteric bacteria (i.e., the bacteria that resides in the human intestinal tract), the same experimental design was applied to Escherichia coli K1 in LB media. CsCl had only a minimal effect on the growth rate of E. coli K12, even at concentrations as high as 100 mM. The doubling times for all concentrations of CsCl tested (i.e., 0 mM, 5 mM, 10 mM, 25 mM, 50 mM, 75 mM, and 100 mM) were within 15 min or one another, at approximately 1 h.

S. mutans cultures were grown as biofilms on glass slides. Cells were allowed to adhere to the slides before exposure to cesium chloride. After two days of additional growth, biofilms were scraped and quantified after freeze-drying to remove all water. No discernable pattern could be deduced from these experiments. All cultures had similar dry weight measurements, though variability was high.

It was hypothesized that, as a larger monovalent cation, cesium could block potassium channels, thereby blocking the uptake of potassium into the cell and retarding cell growth. Strains of E. coli with non-essential genes knocked out were acquired from the Yale Coli Genetic Stock Center. Four mutants were chosen: JW0686, a KdpA mutant, a cesium inducible potassium transporting ATPase; JW1242, a Kch mutant, a conductive potassium transporter; JW1358, a TrkG mutant, a transmembrane component of the Trk potassium uptake system; JW5576, a TrkH mutant, a transmembrane component of the Trk potassium uptake system. TrkG and TrkH determine the specificity of the Trk protein. Because of their similarity (i.e., 41% nucleotide homology), Trk can be made with either one of the two subunits if the other is not available, although TrkG is preferred. All mutants exhibited some inhibition by CsCl, but wild type E. coli K-12 did not.

One explanation for the observations above is that the CsCl was blocking potassium channels. Since E. Coli possesses at least four potassium channels, removing one only slight retards its ability to take up potassium. This is consistent with experimental observations: a roughly one third reduction in growth rate between the control with 100 mM NaCl and the most inhibited cultures at 100 mM CsCl. With one less potassium channel, the remaining channels are more likely to be blocked by cesium at concentrations not previously inhibitory.

The effects of pH on CsCl inhibition were also investigated. S. mutans growth could not be sustained at pH 4.04. At pH 4.50, CsCl was less inhibitory than at neutral or basic pH (pH 7.00 and 8.33 respectively). Previous experiments were carried out at a pH of 7.25 and demonstrated an intermediate inhibition between pH 7.00 and 8.33. The most basic pH tested, pH 8.33, showed the strongest inhibition. No inhibition had been previously seen with 25 mM CsCl at pH 7.25; however, the doubling time of cultures increased by 25% at pH 8.33, from 52 minutes at pH 7.00 to 64 minutes at pH 8.33. Cesium chloride (100 mM) was more inhibitory at pH 8.33 that at neutral pH.

Alternate cesium salts were tested to determine their affect on S. mutans growth. Cesium salts of hydroxide, formate, bicarbonate, sulfate, nitrate, fluoride and iodide were tested at 0 mM, 25 mM, 50 mM and 100 mM concentrations. Cesium formate, fluoride and iodide showed little to no inhibition at the concentrations tested. Cesium sulfate (CsSO₄) was not inhibitory at concentrations of 50 mM or below. Cesium nitrate (CsNO₃) inhibited culture growth rates by roughly 30%, which was the same as for control (sodium nitrate, NaNO₃). Cesium hydroxide (CsOH) inhibited cultures completely at 50 mM or greater due to increases in pH rather than increased cesium effect, as demonstrated by a sodium hydroxide control. CsOH and NaOH increased the pH of the media to 9.5 and 12.5 at 50 mM and 100 mM respectively, which is outside the range of S. mutans growth.

Cesium bicarbonate (CsHCO₃) demonstrated increased inhibition at 50 mM and complete inhibition of S. mutans growth at 100 mM. While inhibition at concentrations less than 45 mM was not significantly different that for sodium bicarbonate control, concentrations of 45 mM cesium bicarbonate or greater inhibited S. mutans growth more than the control cultures. A Lineweaver-Burke plot indicated that the MIC of cesium bicarbonate is 40.5 mM, more than 20 mM lower than the MIC for CsCl. A previous study of S. mutans growth in the presence of sodium bicarbonate showed that sodium bicarbonate is completely inhibitory at 8% (% w/v, ˜950 mM), a concentration well above the complete inhibition seen at 100 mM CsHCO₃.

In further experiments, S. mutans was grown to early exponential phase before spiking cultures with 100 mM CsCl. After 3 hours of incubation, the cultures were spiked with either 100 mM NaCl, 100 mM CsCl or Listerine (1% dilution or a 10% dilution in the media). The NaCl spiked culture grew with a doubling time of 1.21 h, while the CsCl culture grew with a doubling time or 2.49 h.

Listerine was chosen to compare the antimicrobial effects of CsCl to a marketed product. The 10% Listerine in TS media showed similar growth to the 100 mM CsCl with a doubling time of 2.07 hour. The 1% Listerine dilution, however, exhibited a faster doubling time than any of the other cultures at 1.16 hour and grew to a higher yield of 1.2 OD units (as compared to a doubling time of 1.21 hours and a yield of 0.9 OD units for the NaCl control).

1. A composition for treating oral disease or oral pain, wherein the composition comprises: cesium bicarbonate, rubidium bicarbonate, or a mixture of cesium bicarbonate and rubidium bicarbonate at a concentration between 40 mM and 110 mM in an aqueous solution; and wherein the composition has a pH between 7.0 and 9.0.

2. The composition according to claim 1, wherein the composition comprises cesium bicarbonate.

3. The composition according to claim 2, wherein the cesium bicarbonate is at a concentration between 40 mM and 75 mM.

4. The composition according to claim 3, wherein the composition has a pH between 8.0 and 9.0.

5. The composition according to claim 4, wherein the cesium bicarbonate is at a concentration between 45 mM and 70 mM.

6. The composition according to claim 5, wherein the composition has a pH between 8.25 and 8.5.

7. A composition for treating oral disease or oral pain, wherein the composition comprises: cesium bicarbonate, rubidium bicarbonate, or a mixture of cesium bicarbonate and rubidium bicarbonate at a concentration between 0.5% and 20% weight/weight of a solid or semi-solid; and wherein the composition is in the form of a lozenge or gum.

8. The composition according to claim 7, wherein the composition comprises cesium bicarbonate in the form of a lozenge.

9. The composition according to claim 8, wherein the composition further comprises sodium bicarbonate.

10. The composition according to claim 9, wherein the composition further comprises menthol and sucralose.

11. The composition according to claim 10, wherein the composition further comprises xylitol, ethyl cellulose polymer, gum Arabic and peppermint oil.

12. The composition according to claim 11, wherein the composition further comprises eucalyptus oil, thymol, wintergreen and glycerol.

13. A method of treating an oral disease, wherein the method comprises the step of bringing a composition in contact with one or more oral tissues of a patient, and wherein the composition comprises: cesium bicarbonate, rubidium bicarbonate, or a mixture of cesium bicarbonate and rubidium bicarbonate at a concentration between 40 mM and 110 mM in an aqueous solution; and wherein the composition has a pH between 7.0 and 9.0.

14. The method according to claim 13, wherein the composition comprises cesium bicarbonate.

15. The method according to claim 14, wherein the cesium bicarbonate is at a concentration between 40 mM and 75 mM.

16. The method according to claim 15, wherein the composition has a pH between 8.0 and 9.0.

17. The method according to claim 16, wherein the cesium bicarbonate is at a concentration between 45 mM and 70 mM.

18. The method according to claim 17, wherein the composition has a pH between 8.25 and 8.5.

19. The method according to claim 18, wherein the composition is in the form of an oral rinse.

20. The method according to claim 19, wherein the cesium bicarbonate is at a concentration of about 50 mM.
